(19) United States
(12) Patent Application Publication
(10) Pub. No.: US 2012/0234204 A1
(43) Pub. Date: Sep. 20, 2012

(54) VAPOR PHASE FUNCTIONALIZATION OF CARBON NANOTUBES

(75) Inventors: Serkan Unal, Pittsburgh, PA (US); Robert N. Hunt, Steubenville, OH (US); Heiko Hocke, Leverkusen (DE)

(73) Assignees: Bayer MaterialScience AG, Leverkusen (DE); Bayer MaterialScience LLC, Pittsburgh, PA (US)

(21) Appl. No.: 13/050,199

(22) Filed: Mar. 17, 2011

(51) Int. Cl. C08L 1/12; C08K 5/20; C07C 231/02; C07C 233/78; B82Y 30/00

(52) U.S. Cl. 106/170.44; 564/139; 564/156; 524/227; 524/229; 524/228; 977/746

(57) ABSTRACT

The present invention provides a process for the production of functionalized carbon nanotubes. The inventive process for the production of functionalized carbon nanotubes involves reacting carboxylic acid moieties of oxidized carbon nanotubes with vapors of a compound containing carboxylic acid reactive groups to produce functionalized carbon nanotubes. The present invention also provides a composition made from a plastic resin and functionalized carbon nanotubes produced by reacting carboxylic acid moieties of oxidized carbon nanotubes with a vapor containing carboxylic acid reactive groups to produce functionalized carbon nanotubes. The inventive process is useful with single-walled carbon nanotubes, double-walled carbon nanotubes and multi-walled carbon nanotubes.

### FIELD OF THE INVENTION

The present invention relates in general to carbon nanotubes, and more specifically, to a vapor phase process for the functionalization of carbon nanotubes.

### BACKGROUND OF THE INVENTION

Carbon nanotubes have been known in the literature for almost two decades. Iijima (Nature, 354, 56-58, 1991) is generally considered to have discovered nanotubes, however, such materials as fibrous graphite materials having a plurality of graphite layers, have been known since the 1970's or early 1980's. Carbon nanotubes (also sometimes referred to as carbon fibrils) are seamless tubes of graphite sheets with full fullerene caps which were first discovered as multi-layer concentric tubes or multi-walled carbon nanotubes and subsequently as single-walled carbon nanotubes in the presence of transition metal catalysts. Carbon nanotubes have shown promise in applications including nanoscale electronic devices, high strength materials, electron field emission, tips for scanning probe microscopy, and gas storage.

Various processes and catalysts are known in the art for the production of carbon nanotubes. An overview of production methods is given, for example, by Geus and DeJong in a review article (K. P. De Jong and J. W. Geus in Catal. Rev.—Sci. Eng., 42(4), 2000, pages 481-510). Either pure metals or combinations of various metals can be employed, as described e.g. in WO 03/004410 A1, U.S. Pat. No. 6,358,878, U.S. Pat. No. 6,518,218, and CN 1443708.

The functionalization of carbon nanotubes has become a very important step for their successful incorporation into polymeric matrices. Two major approaches for functionalizing carbon nanotubes have been tried to date: oxidation and amine functionalization.

The first approach to the functionalization of carbon nanotubes involves ozone oxidation or ultraviolet/ozone treatment, such as in U.S. Pat. No. 7,122,165, issued to Wong et al. The '165 patent discloses a method of functionalizing the sidewalls of a plurality of carbon nanotubes with oxygen moieties, involving exposing a carbon nanotube dispersion to an ozone/oxygen mixture to form a plurality of ozonized carbon nanotubes and contacting the plurality of ozonized carbon nanotubes with a cleaving agent to form a plurality of sidewall-functionalized carbon nanotubes.

Ziegler et al, in U.S. Pat. No. 7,470,417, describe methods of ozonating CNTs in fluorinated solvents (fluoro-solvents). Such methods are said to provide a less dangerous alternative to existing ozonolysis methods. In some embodiments, the methods involve the steps of: (a) dispersing carbon nanotubes in a fluoro-solvent to form a dispersion; and (b) reacting ozone with the carbon nanotubes in the dispersion to functionalize the sidewalls of the carbon nanotubes and yield functionalized carbon nanotubes with oxygen-containing functional moieties. In some embodiments, the fluoro-solvent is a fluorocarbon solvent, such as a perfluorinated polyether.

U.S. Pat. No. 7,413,723 issued to Niu et al., provides methods of oxidizing multi-walled carbon nanotubes. The multi-walled carbon nanotubes are oxidized by contacting the carbon nanotubes with gas-phase oxidizing agents such as CO2, O2, steam, N2O, NO, NO2, O3, and ClO2. Near critical and supercritical water are said to be useful as oxidizing agents. The multi-walled carbon nanotubes oxidized according to methods of the invention of Niu et al., are said to be useful in the preparation of rigid porous structures which can be utilized to form electrodes for fabrication of improved electrochemical capacitors.

Ma et al., in U.S. Published Patent Application No. 2008/0031802, describe methods of treating single walled and multi-walled carbon nanotubes with ozone. The carbon nanotubes are treated by contacting the carbon nanotubes with ozone at a temperature range between 0° C. and 100° C. to yield functionalized nanotubes which are greater in weight than the untreated carbon nanotubes. The carbon nanotubes treated according to methods of Ma et al. are said to be useful in the preparation of complex structures such as three dimensional networks or rigid porous structures which can be utilized to form electrodes for fabrication of improved electrochemical capacitors. Useful catalyst supports are said to be prepared by contacting carbon nanotube structures such as carbon nanotube aggregates, three dimensional networks or rigid porous structures with ozone in the temperature range between 0° C. and 100° C.

U.S. Published Patent Application No. 2008/0102020, in the name of Niu et al., discloses methods of oxidizing multi-walled carbon nanotubes. The multi-walled carbon nanotubes are oxidized by contacting the carbon nanotubes with gas-phase oxidizing agents such as CO2, O2, steam, N2O, NO, NO2, O3, and ClO2. Niu et al. state near critical and supercritical water can also be used as oxidizing agents. The multi-walled carbon nanotubes oxidized according to methods of Niu et al. are said to be useful in the preparation of rigid porous structures which can be utilized to form electrodes for fabrication of improved electrochemical capacitors.

Juni et al., in U.S. Published Patent Application No. 2008/0152573, provide a method for producing dispersible carbon nanotubes by subjecting single-walled carbon nanotubes to UV treatment in the presence of ozone such that carboxyl groups are introduced into the single-walled carbon nanotubes and that the single-walled carbon nanotubes are fragmented. Carbon nanotubes obtained by the method of Juni et al. are said to have good dispersibility.

Published PCT Application No. WO 2001/07694, in the name of Niu et al., describes methods of oxidizing multi-walled carbon nanotubes. The multi-walled carbon nanotubes are oxidized by contacting the carbon nanotubes with gas-phase oxidizing agents such as CO2, O2, steam, N2O, NO, NO2, O3, and ClO2. Near critical and supercritical water can also be used as oxidizing agents. The multi-walled carbon nanotubes oxidized according to methods of Niu et al. are said to be useful in the preparation of rigid porous structures which can be utilized to form electrodes for fabrication of improved electrochemical capacitors.

The second of approach involves amino functionalization of carbon nanotubes. For example, U.S. Pat. No. 6,099,960, issued to Tennent, et al., discloses a high surface area carbon nanofiber. The carbon nanofiber has an outer surface on which a porous high surface area layer is formed. A method of making the high surface area carbon nanofiber includes pyrolizing a polymeric coating substance provided on the outer surface of the carbon nanofiber at a temperature below the temperature at which the polymeric coating substance melts. The polymeric coating substance used as the high surface area around the carbon nanofiber may include phenolics-formaldehyde, polyacrylonitrile, styrene, divinyl benzene, cellulosic polymers and cyclotrimerized diethynyl benzene. The high surface area polymer which covers the carbon nanofiber may be functionalized with one or more functional groups.

Haddon et al., in U.S. Pat. No. 6,187,823, describe naked single-walled nanotube carbon metals and semiconductors dissolved in organic solutions by direct functionalization with amines or alkylaryl amines having an uninterrupted carbon chain of at least five and more preferably nine carbon atoms in length.

U.S. Pat. No. 6,203,814, issued to Fisher et al., provides graphitic nanotubes, which include tubular fullerenes (commonly called “buckytubes”) and fibrils, which are functionalized by chemical substitution or by adsorption of functional moieties. The graphitic nanotubes of Fisher et al. are uniformly or non-uniformly substituted with chemical moieties or upon which certain cyclic compounds are adsorbed and to complex structures comprised of such functionalized fibrils linked to one another. Fisher et al., also provide methods of introducing functional groups onto the surface of such fibrils.

Haddon et al., in U.S. Pat. No. 6,368,569, describe naked carbon nanotubes dissolved in organic solutions by terminating the nanotubes with carboxylic acid groups and attaching an aliphatic carbon chain so as to render the carbon nanotubes soluble.

U.S. Pat. No. 6,531,513, issued to Haddon et al., discloses carbon nanotubes dissolved in organic solutions by attaching an aliphatic carbon chain (which may contain aromatic residues) so as to render the carbon nanotubes soluble.

Niu et al., in U.S. Pat. Nos. 6,872,681 and 7,070,753, provide a method of chemically modifying carbon nanotubes having a diameter less than one micron involving contacting the nanotubes with a peroxygen compound selected from organic peroxyacids, inorganic peroxyacids and organic hydroperoxides, or a salt thereof, under oxidation conditions and thereby producing modified carbon nanotubes. Oxidation of the nanotubes is said by Niu et al. to increase the degree of dispersion of aggregates of nanotubes and aid in the disassembling of such aggregates. The dispersed nanotubes are said to be useful in the preparation of rigid structures for use in electrodes and capacitors.

Khabashesku et al., in U.S. Pat. No. 7,125,533, disclose a method for functionalizing the wall of single-wall or multi-wall carbon nanotubes which involves the use of acyl peroxides to generate carbon-centered free radicals. The method is said to allow for the chemical attachment of a variety of functional groups to the wall or end cap of carbon nanotubes through covalent carbon bonds without destroying the wall or endcap structure of the nanotube. According to Khabashesku et al., carbon-centered radicals generated from acyl peroxides can have terminal functional groups to provide sites for further reaction with other compounds. Organic groups with terminal carboxylic acid functionality can be converted to an acyl chloride and further reacted with an amine to form an amide or with a diamine to form an amide with terminal amine. The reactive functional groups attached to the nanotubes are said to provide improved solvent dispersibility and provide reaction sites for monomers for incorporation in polymer structures. Khabashesku et al. state the nanotubes can also be functionalized by generating free radicals from organic sulfoxides.

U.S. Pat. No. 7,276,283, issued to Denes et al., provides methods for producing plasma-treated, functionalized carbon-containing surfaces. The methods include the steps of subjecting a carbon-containing substrate to a plasma to create surface active sites on the surface of the substrate and reacting the surface active sites with stable spacer molecules in the absence of plasma. Biomolecules may be immobilized on the resulting functionalized surfaces. The methods of Denes et al. are said to be useful in treating a variety of carbon-containing substrates, including polymeric surfaces, diamond-like carbon films and carbon nanotubes and nanoparticles.

U.S. Pat. No. 7,452,519, issued to Khabashesku et al., discloses a method of sidewall-functionalizing single-walled carbon nanotubes through C—N bond forming substitution reactions with fluorinated single-walled carbon nanotubes (fluoronanotubes), and sidewall-functionalized single-walled carbon nanotubes containing C—N bonds between carbons of the single-walled carbon nanotube sidewall and nitrogen atoms of the functionalizing groups. Where diamine species are utilized as reactants, novel materials like crosslinked single-walled carbon nanotubes and “nanotube-nylons” are said to be generated. In some embodiments, single-walled carbon nanotubes with functional groups covalently attached to their side walls through C—N bonds are prepared by either the direct interaction of fluoronanotubes with terminal alkylidene diamines or diethanolamine, or by a two-step procedure involving consecutive treatments with Li3N in diglyme and RCl(R═H, n-butyl, benzyl) reagents. Sidewall attachment of amine-derived groups is said to be provided by Raman, FTIR, and UV-vis-NIR spectra, SEM/EDAX and TEM data, and thermal degradation studies. The C—N functionalization methods of Khabashesku et al. are said to offer a wide range of further single-walled carbon nanotube derivatizations, including their covalent binding to amino acids, DNA, and polymer matrixes.

U.S. Published Patent Application Nos. 2006/0249711 and 2008/0176983, both in the name of Niu et al., describe polymer composites composed of a polymerized mixture of functionalized carbon nanotubes and monomer which chemically reacts with the functionalized nanotubes. The carbon nanotubes are functionalized by reacting with oxidizing or other chemical media through chemical reactions or physical adsorption. The reacted surface carbons of the nanotubes are further functionalized with chemical moieties that react with the surface carbons and selected monomers. The functionalized nanotubes are first dispersed in an appropriate medium such as water, alcohol or a liquefied monomer and then the mixture is polymerized. The polymerization results in polymer chains of increasing weight bound to the surface carbons of the nanotubes. The composite may consist of some polymer chains imbedded in the composite without attachment to the nanotubes. The resulting composite is said to yield superior chemical, physical and electrical properties over polymer composites that are only physically mixed and without binding to the surface carbons of the nanotubes.

U.S. Published Patent Application No. 2009/0124747, in the name of Khabashesku et al., describes carbon nanotube materials and condensation polymers having at least one bridge between carbon nanotubes. Carbon nanotube materials comprise a plurality of functionalized single-wall carbon nanotubes linked to at least one other single-wall carbon nanotube by at least one bridge. The at least one bridge comprises at least one amine functionality bonded to the functionalized single-wall carbon nanotubes. The amine functionality may be alkyl or aryl. Khabashesku et al. also disclose carbon nanotube condensation polymers having at least one bridge between single-wall carbon nanotubes. The bridges in the condensation polymers contain an amine functionality and a condensation agent.

A need continues to exist in the art for improved processes of functionalizing single-walled or multi-walled carbon nanotubes to aid in the incorporation of those materials into polymers.

### SUMMARY OF THE INVENTION

Accordingly, the present invention provides a process for the production of functionalized carbon nanotubes. The inventive process for the production of functionalized carbon nanotubes involves reacting carboxylic acid moieties of oxidized carbon nanotubes with vapors of a compound containing carboxylic acid reactive groups to produce functionalized carbon nanotubes. The present invention also provides a composition made from aplastic resin and functionalized carbon nanotubes produced by reacting carboxylic acid moieties of oxidized carbon nanotubes with a vapor containing carboxylic acid reactive groups to produce functionalized carbon nanotubes. The inventive process is useful with single-walled carbon nanotubes, double-walled carbon nanotubes and multi-walled carbon nanotubes.

These and other advantages and benefits of the present invention will be apparent from the Detailed Description of the Invention herein below.

### BRIEF DESCRIPTION OF THE FIGURES

The present invention will now be described for purposes of illustration and not limitation in conjunction with the figure, wherein:

FIG. 1 illustrates one embodiment of the inventive process;

FIG. 2 shows a spectroscopic analysis of the final products of Examples 1 and 2 using Fourier Transform Infrared Spectroscopy (FT-IR); and

FIG. 3 provides a FT-IR spectroscopic analysis of the final product from Example 4.

### DETAILED DESCRIPTION OF THE INVENTION

The present invention will now be described for purposes of illustration and not limitation. Except in the operating examples, or where otherwise indicated, all numbers expressing quantities, percentages, and so forth in the specification are to be understood as being modified in all instances by the term “about.”

The present invention provides a process for the production of functionalized carbon nanotubes involving reacting carboxylic acid moieties of oxidized carbon nanotubes with vapors of a compound containing carboxylic acid reactive groups to produce functionalized carbon nanotubes.

The oxidized carbon nanotubes useful in the present invention may be produced by any process known to those skilled in the art, for example by oxidizing the carbon nanotubes with ozone, by oxidizing the carbon nanotubes in a UV-ozone fluidized bed reactor, and by oxidizing the carbon nanotubes with nitric acid and/or sulfuric acid.

The present invention further provides a composition made from a plastic resin and functionalized carbon nanotubes produced by reacting carboxylic acid moieties of oxidized carbon nanotubes with a vapor containing carboxylic acid reactive groups to produce functionalized carbon nanotubes.

The present invention is useful with any type of carbon nanotubes, including, but not limited to, single-walled carbon nanotubes, double-walled carbon nanotubes and multi-walled carbon nanotubes. Single-walled carbon nanotubes suitable for use in the present invention can be produced by a variety of methods known to those skilled in the art. Some preferred processes are described in U.S. Pat. Nos. 6,827,919; 6,221,330; 7,074,379; 7,097,821; 7,144,564; and 7,862,795 and in U.S. Published Patent Application Nos. 2008/0175786, 2008/0279751, 2010/0086472 and 2010/0221173.

As those skilled in the art are aware, multi-walled carbon nanotubes may be produced by a variety of methods. Some preferred processes are described in U.S. Published Patent Application Nos. 2008/0003170, 2008/0293853, 2009/0023851, 2009/0124705 and 2009/0140215.

The present invention requires a method of introducing carboxylic acid moieties on the surface of the single-walled or multi-walled carbon nanotubes. Such methods include a fluidized bed process which may take as little as one to three hours. An advantage to the fluidized bed process is that the nanotubes will remain in their aggregate granular form as supplied by the carbon nanotube manufacturer, thereby reducing the necessity of handling fine carbon nanotube powders. The nanotubes also will remain in a dry state and are ready for dispersing in the desired liquid without any further processing. The inventive process eliminates the need for refluxing or sonicating with concentrated acid and the required steps of neutralizing, washing, filtering and waste disposal required with traditional carbon nanotube surface activation. The inventive process can be easily scaled up for commercial production. Another acceptable method of introducing carboxylic acid moieties is to oxidize carbon nanotubes in nitric acid and/or sulfuric acid.

Although described herein with ethylenediamine, other functionalizing agents such as diethylenetriamine, triethylenetetraamine, 1,4-butanediamine, 1,6-hexanediamine, 2-methypentamethylenediamine, isophorone diamine, diethanolamine, ethanolamine may be used. The present inventors believe the approach described herein can be extended to any compound having a carboxylic acid reactive group as long as it can be vaporized at 150-200° C. under atmospheric pressure or under vacuum. As those skilled in the art will appreciate, single-walled and multi-walled carbon nanotubes functionalized by the inventive process can be covalently incorporated into a variety of plastic resins.

Suitable plastic resins include, but are not limited to, acrylonitrile butadiene styrene (ABS), polymethylmethacrylate (PMMA), cellulose acetate, cycloolefin copolymer (COC), ethylene-vinyl acetate (EVA), ethylene vinyl alcohol (EVOH), fluoroplastics, such as polytetrafluoroethylene (PTFE), fluorinated ethylene propylene (FEP), chlorotrifluoroethylene (CTFE), ethylene chlorotrifluoroethylene (ECTFE), ethylene tetrafluoroethylene (ETFE), polyacetal, polyacrylates, polyacrylonitrile (PAN), polyamide (PA or Nylon), polyamide-imide (PAI), polyaryletherketone (PAEK), polybutadiene (PBD), polybutylene (PB), polybutylene terephthalate (PBT), polycaprolactone (PCL), polychlorotrifluoroethylene (PCTFE), polyethylene terephthalate (PET), polycyclohexylene dimethylene terephthalate (PCT), polycarbonate (PC), polyhydroxyalkanoates (PHAs), polyester, polyethylene (PE), polyetheretherketone (PEEK), polyetherketoneketone (PEKK), polyetherimide (PEI), polyethersulfone (PES), polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polytrimethylene terephthalate (PTT), polyurethane (PU), polyvinyl acetate (PVA), polyvinyl chloride (PVC), polyvinylidene chloride (PVDC), and styrene-acrylonitrile (SAN).

### EXAMPLES

The present invention is further illustrated, but is not to be limited, by the following examples.

In the examples below, multi-walled carbon nanotubes were oxidized in a UV-ozone fluidized bed reactor to introduce carboxylic acid moieties on the surface of the nanotubes. Such UV-ozone treatments are known to those skilled in the art (See, e.g., Najafi, E. et al., “UV-ozone treatment of multi-walled carbon nanotubes for enhanced organic solvent dispersion”, Colloids and Surfaces A: Physicochem Eng. Aspects, 284-285 (2006) 373-378; Parkekh, B. et al., “Surface functionalization of multiwalled carbon nanotubes with UV and vacuum UV photo-oxidation”, J. Adhesion Sci. Technol. Vol. 20, No. 16, pp. 1833-1846 (2006); and Simmons, J. M. et al., “Effect of Ozone Oxidation on Single-Walled Carbon Nanotubes”, J. Phys. Chem. B 2006, 110, 7113-7118).

Briefly, a gaseous oxidation ozone gas combined with UV light and was passed through a column containing multi-walled carbon nanotubes. The UV-ozone treated carbon nanotubes were subsequently reacted with ethylenediamine (EDA) vapor at 155-160° C. for five hours to introduce amine groups via the condensation reaction between carboxylic acid and amine groups. Following the condensation reaction, the products were vacuum dried to remove unreacted, residual ethylenediamine and water on the surface of the nanotubes.

The present inventors recognize the above-described is not the only useful process to oxidize carbon nanotubes prior to vapor phase amino-functionalization. Another acceptable method is to oxidize carbon nanotubes in nitric acid and/or sulfuric acid. There are numerous publications detailing this approach in the literature. Further, as will be readily apparent to those skilled in the art, any oxidized carbon nanotubes containing carboxylic acid moieties are useful in the present invention.

Although the present invention is described as amino functionalization, those skilled in the art will appreciate other carboxylic acid reactive group can be used to functionalize the oxidized carbon nanotubes. Some examples of such reactive groups include, alcohols, phosphorous trichloride (PCl3), phosphorous pentachloride (PCl5), thionyl chloride (SOCl2), and phosphorous tribromide (PBr3).

### Example 1

Multi-walled carbon nanotubes (4.0 g, BAYTUBES C 150 P) were oxidized in a fluidized bed reactor, in which a combination of ozone gas with oxygen radicals generated by a UV-light was passed through the multi-walled carbon nanotubes for eight hours to introduce carboxylic acid moieties on the carbon nanotube surface.

### Example 2

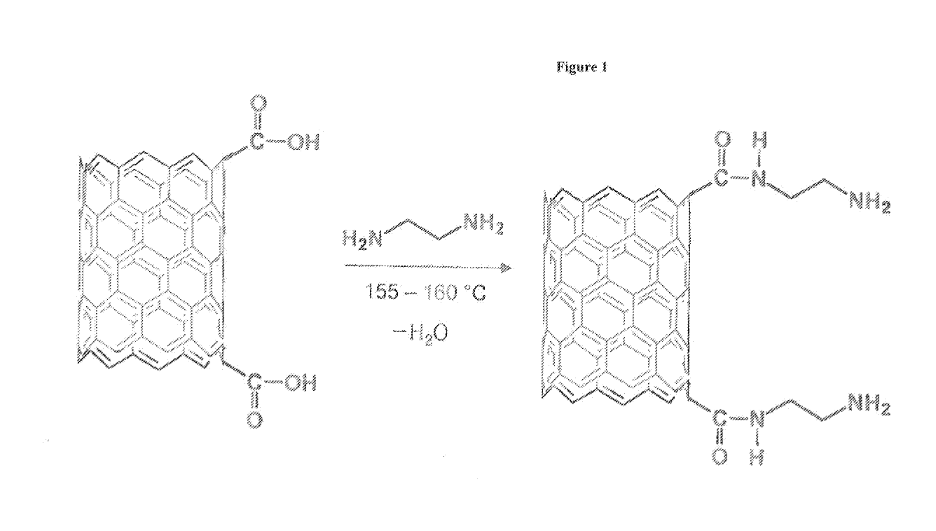

Oxidized multi-walled carbon nanotubes from Example 1 (4.0 g) were placed in a column that was heated to 155-160° C. and vapors of ethylenediamine were passed through the column continuously in a reaction apparatus, in which ethylenediamine was circulated by refluxing in a three-neck flask, driving ethylenediamine vapors through the column containing oxidized multi-walled carbon nanotubes and returning into the three-neck flask by condensing in a condenser that is attached to both the column and the flask. The reaction was maintained for five hours to facilitate a condensation reaction between carboxylic acid and amine groups to form an amide linkage as shown in FIG. 1. Following the treatment with saturated ethylenediamine vapors, the functionalized multi-walled carbon nanotubes were vacuum dried at 0.015 torr at 163° C. for 20 minutes.

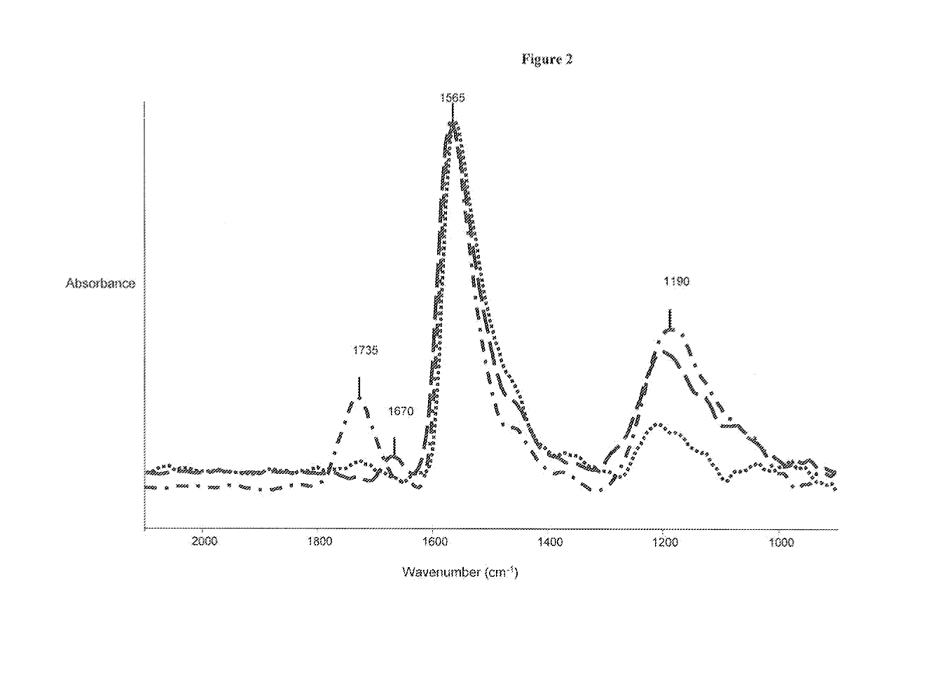

As shown in FIG. 2, spectroscopic analysis of the final products of Examples 1 (dotted dashed line) and 2 (dashed line) using Fourier Transform Infrared Spectroscopy (FT-IR) revealed non-oxidized multi-walled carbon nanotubes (dotted line) showed a strong absorbance at approximately 1565 cm-1 due to aromatic C═C stretching. Upon oxidation of multi-walled carbon nanotubes as described in Example 1, a peak was observed at approximately 1565 cm-1 due to carbonyl (C═O) stretching from carboxylic acid moieties. Upon amino-functionalization of the oxidized multi-walled carbon nanotubes as described in Example 2, the carboxylic acid carbonyl stretching peak shifted to 1670 cm-1, which the present inventors attributed to amide carbonyl stretching (see structures in FIG. 1). Note: the present inventors attributed the peak at 1190-1200 cm-1 to zinc selenide, which was used as the substrate for the FT-IR analysis.

### Example 3

Multi-walled carbon nanotubes (4.0 g, BAYTUBES C 150 P), without oxidation, were treated with saturated ethylenediamine vapors in the same apparatus as used in Example 2 under the same conditions. Following the treatment with saturated ethylenediamine vapors, the multi-walled carbon nanotubes were vacuum dried for 20 minutes at 0.010 torr at 163° C.

Surface analysis of the final products from Examples 1-3 for atomic content using x-ray photo-electron spectroscopy (XPS) revealed enrichment of the surface with nitrogen atoms after this process as can be appreciated by reference to Table I.

TABLE I

| Sample | C % | O % | N % |
|---|---|---|---|
| Multi-walled carbon nanotubes | 99.3 | 0.7 | — |
| Example 1 | 91.9 | 7.8 | 0.2 |
| Example 2 | 90.6 | 5.9 | 3.4 |
| Example 3 | 96.0 | 2.7 | 1.3 |

### Example 4

Multi-walled carbon nanotubes (100 g, BAYTUBES C 70 P) were oxidized by combining with 2,500 g of 8 M nitric acid in a five-liter round-bottom flask and stirring for 92 hours under nitrogen purge at room temperature. The multi-walled carbon nanotubes were filtered out and washed extensively with water and water/isopropanol mixture, and then dried under vacuum at 90° C. for three days.

The dried, oxidized multi-walled carbon nanotubes (54.5 g) were placed in a column that was heated to 180° C. and vapors of diethylenetriamine were passed through the column continuously in a reaction apparatus, in which diethylenetriamine was circulated by refluxing under 225 torr vacuum at 170° C. in a three-neck flask. This apparatus drove the diethylenetriamine vapors through the column containing oxidized multi-walled carbon nanotubes and returned them into the three-neck flask by condensing in a condenser attached to both the column and the flask. The reaction was maintained for 12 hours to facilitate a condensation reaction. Following treatment with saturated diethylenetriamine vapors, the functionalized multi-walled carbon nanotubes were subjected to extensive washing with acetone and water and then vacuum dried at 90° C. for three days.

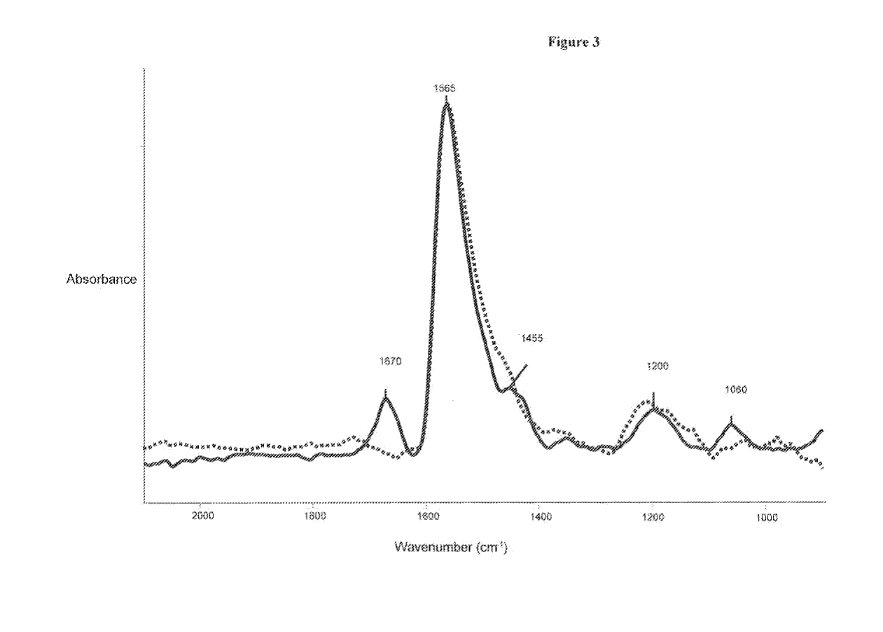

As shown in FIG. 3, FT-IR spectroscopic analysis of the final product from Example 4 (solid line) clearly shows functionalization of the non-oxidized multi-walled carbon nanotubes (dotted line) was achieved, as a peak appeared at 1670 cm-1 attributed to carbonyl (C═O) stretching of an amide linkage. In addition, weaker bending peaks were also observed such as the shoulder at 1453 cm-1 attributed to —CH2- bending and the peak at 1061 cm-1 attributed to —NH2 bending.

The foregoing examples of the present invention are offered for the purpose of illustration and not limitation. It will be apparent to those skilled in the art that the embodiments described herein may be modified or revised in various ways without departing from the spirit and scope of the invention. The scope of the invention is to be measured by the appended claims.

What is claimed is:

1. A process for the production of functionalized carbon nanotubes comprising:
reacting carboxylic acid moieties of oxidized carbon nanotubes with vapors of a compound containing carboxylic acid reactive groups to produce functionalized carbon nanotubes.

2. The process according to claim 1, wherein the oxidized carbon nanotubes containing carboxylic acid moieties on the surface of the nanotubes are produced by oxidizing the carbon nanotubes with ozone.

3. The process according to claim 1, wherein the oxidized carbon nanotubes containing carboxylic acid moieties on the surface of the nanotubes are produced by oxidizing the carbon nanotubes in a UV-ozone fluidized bed reactor.

4. The process according to claim 1, wherein the oxidized carbon nanotubes containing carboxylic acid moieties on the surface of the nanotubes are produced by oxidizing the carbon nanotubes with nitric acid and/or sulfuric acid.

5. The process according to claim 1, wherein the carbon nanotubes are selected from the group consisting of single-walled, double-walled and multi-walled.

6. The process according to claim 1, wherein the carboxylic acid reactive group is selected from the group consisting of amines, alcohols, phosphorous trichloride (PCl3), phosphorous pentachloride (PCl5), thionyl chloride (SOCl2), and phosphorous tribromide (PBr3).

7. The process according to claim 1, where in the carboxylic acid reactive group is an amine selected from the group consisting of ethylenediamine, diethylenetriamine, triethylenetetraamine, 1,4-butanediamine, 1,6-hexanediamine, 2-methypentamethylenediamine, isophorone diamine, diethanolamine and ethanolamine.

8. Functionalized carbon nanotubes produced by the process according to claim 1.

9. Functionalized carbon nanotubes produced by the process according to claim 2.

10. Functionalized carbon nanotubes produced by the process according to claim 3.

11. Functionalized carbon nanotubes produced by the process according to claim 4.

12. A composition comprising:
a plastic resin; and
functionalized carbon nanotubes produced by reacting carboxylic acid moieties of oxidized carbon nanotubes with a vapor containing carboxylic acid reactive groups to produce functionalized carbon nanotubes.

13. The composition according to claim 12, wherein the oxidized carbon nanotubes containing carboxylic acid moieties on the surface of the nanotubes are produced by oxidizing carbon nanotubes with ozone.

14. The composition according to claim 12, wherein the oxidized carbon nanotubes containing carboxylic acid moieties on the surface of the nanotubes are produced by oxidizing carbon nanotubes in a UV-ozone fluidized bed reactor.

15. The composition according to claim 12, wherein the oxidized carbon nanotubes containing carboxylic acid moieties on the surface of the nanotubes are produced by oxidizing carbon nanotubes with nitric acid and/or sulfuric acid.

16. The composition according to claim 12, wherein the carbon nanotubes are selected from the group consisting of single-walled, double-walled and multi-walled.

17. The composition according to claim 12, wherein the carboxylic acid reactive group is selected from the group consisting of amines, alcohols, phosphorous trichloride (PCl3), phosphorous pentachloride (PCl5), thionyl chloride (SOCl2), and phosphorous tribromide (PBr3).

18. The composition according to claim 12, where in the carboxylic acid reactive group is an amine selected from the group consisting of ethylenediamine, diethylenetriamine, triethylenetetraamine, 1,4-butanediamine, 1,6-hexanediamine; 2-methypentamethylenediamine, isophorone diamine, diethanolamine and ethanolamine.

19. The composition according to claim 12, wherein the resin is selected from the group consisting of acrylonitrile butadiene styrene, polymethylmethacrylate, cellulose acetate, cycloolefin copolymer, ethylene-vinyl acetate, ethylene vinyl alcohol, polytetrafluoroethylene, fluorinated ethylene propylene, chlorotrifluoroethylene, ethylene chlorotrifluoroethylene, ethylene tetrafluoroethylene, polyacetal, polyacrylates, polyacrylonitrile, polyamide, polyamide-imide, polyaryletherketone, polybutadiene, polybutylene, polybutylene terephthalate, polycaprolactone, polychlorotrifluoroethylene, polyethylene terephthalate, polycyclohexylene dimethylene terephthalate, polycarbonate, polyhydroxyalkanoates, polyester, polyethylene, polyetheretherketone, polyetherketoneketone, polyetherimide, polyethersulfone, polyethylenechlorinates, polyimide, polylactic acid, polymethylpentene, polyphenylene oxide, polyphenylene sulfide, polyphthalamide, polypropylene, polystyrene, polysulfone, polytrimethylene terephthalate, polyurethane, polyvinyl acetate, polyvinyl chloride, polyvinylidene chloride, and styrene-acrylonitrile.
